(19) United States
(12) Patent Application Publication
(10) Pub. No.: US 2023/0096203 A1
(43) Pub. Date: Mar. 30, 2023

(54) METHODS FOR DETECTING FOOD ALLERGIES

(71) Applicant: THE REGENTS OF THE UNIVERSITY OF MICHIGAN, Ann Arbor, MI (US)

(72) Inventors: Vicky Garcia Hernandez, Ann Arbor, MI (US); Amnah Yamani, Ann Arbor, MI (US); Asma Nusrat, Ann Arbor, MI (US); Simon Patrick Hogan, Ann Arbor, MI (US)

(21) Appl. No.: 17/795,895

(22) PCT Filed: Feb. 26, 2021
(86) PCT No.: PCT/US21/19801

§ 371 Date: Jul. 28, 2022

Related U.S. Application Data
(60) Provisional application No. 62/982,273, filed on Feb. 27, 2020.

(51) Int. Cl. A61K 31/137; A61P 37/08; G01N 33/68

(52) U.S. Cl. CPC A61K 31/137; A61P 37/08; G01N 33/6893; G01N 2800/24; G01N 2333/705

(57) ABSTRACT

Provided herein are methods for determining the presence of desmoglein-2 protein fragments in a sample obtained from a subject suspected of having a food allergy, as well as methods of treating a food allergy.

### CROSS-REFERENCE TO RELATED APPLICATIONS

This application claims the benefit of U.S. Provisional Patent Application No. 62/982,273, filed Feb. 27, 2020, the contents of which are incorporated by reference herein.

### STATEMENT REGARDING GOVERNMENT SUPPORT

This invention was made with government support under A1073553, AI112626, AI138177 and DK090119 awarded by the National Institutes of Health, and under W81XWH-15-1-051730 awarded by the U.S. Department of Defense. The government has certain rights in the invention.

### FIELD

The present invention relates to methods for determining the presence of biomarkers that are indicative of food allergies.

### BACKGROUND

Severe food allergy-related reactions, also known as food-triggered anaphylaxis, are serious life threatening reactions responsible for 30,000-120,000 emergency department visits, 2,000-3,000 hospitalizations, and approximately 150 deaths per year in the United States (Sampson et al., Pediatrics, 111: 1601-1608 (2003); and Ross et al., J. Allergy Clin. Immunol., 121: 166-171 (2008)). The onset of symptoms are variable, occurring within seconds to a few hours following exposure to the dietary allergen, and multiple organ systems are often affected, including gastrointestinal (GI), cutaneous, respiratory and cardiovascular (Wang et al., Clin. Exp. Allergy, 37: 651-660 (2007)). Cutaneous symptoms (urticaria and angioedema) are the most common, occurring in approximately 80% of cases. GI symptoms occur in as much as 40% cases, which include cramping, abdominal pain, nausea, emesis, and diarrhea (Sampson et al., The New England Journal of Medicine, 3.27: 380-384 (1992)). Recent clinical data suggests a link between GI manifestations and more severe anaphylactic phenotypes including hypotension and hypoxia (Schrander et al., J. Pediatr. Gastroenterol. Nutr., 10: 189-192 (1990); Troncone et al., Allergy, 49: 142-446 (1994); Van Elburg et al., Pediatr Allergy Immunol, 4: 79-85 (1993); Calvani et al., Pediatric Allergy and Immunology, 22: 813-819 LID—810.1111/j.1399-3038.2011.01200.x [doi] (2011); and Brown, S. G. A., J of Allergy Clin Immunol, 114: 371-376 (2004))

Food-triggered anaphylaxis can encompass a variety of systemic and intestinal symptoms. Murine-based and clinical studies have revealed a role for histamine and HIR and H2R-pathway in the systemic response; however, the molecular processes that regulate the gastrointestinal (GI) response are not as well defined. There is a need for methods of diagnosing and treating food allergies in susceptible individuals.

### BRIEF SUMMARY OF THE INVENTION

The disclosure provides a method comprising: (a) obtaining a sample from a subject suspected of having a food allergy, and (b) determining the presence of one or more fragments of the desmoglein-2 (DSG-2) protein using an immunoassay.

The disclosure also provides a method of treating a food allergy in a subject in need thereof, which method comprises: (a) determining the presence of one or more fragments of the desmoglein-2 (DSG-2) protein in a sample obtained from the subject using an immunoassay; wherein the presence of one or more fragments of the DSG-2 protein indicates that the subject has a food allergy; and (b) administering a therapeutic agent to the subject, whereby the food allergy is treated.

### BRIEF DESCRIPTION OF THE DRAWING(S)

FIG. 1A is a schematic diagram of the experimental regimen used to demonstrate that food antigen exposure is restricted to the SI during a food-induced anaphylactic reaction. FIG. 1B is a graph showing the presence of OVA-fluorescence in the lower GI tract segments of food allergic WT and iIL-9Tg mice following anti-IgE treatment. OVA-sensitized. BALB/c mice were repeatedly challenged with OVA and on the 7th challenge received MC-labelled FLUOSPHERES™ Polystyrene Microspheres. Localization of FLUOSPHERES™ in the GI segments was examined within 60 minutes. iIL-9Tg mice were challenged with anti-IgE or vehicle and received oral FITC-labelled FLUOSPHERES™. Localization of FLUOSPHERES™ in the GI segments was examined within 60 minutes. Data are represented as the mean fluorescence detected in luminal contents of the respective GI segments; n=5 mice per group.

FIG. 2 includes a series of graphs illustrating that antigen challenge induces SI epithelial transcellular, CTIR-dependent Cl⁻, and paracellular barrier dysfunction. Short-circuit current (Isc) baseline is shown in FIG. 2A. FIG. 2B shows the forskolin-induced short-circuit current response of jejunum segments from Vehicle- and OVA-treated BALB/c wild-type (WT) mice within 60 minutes of the 7th OVA challenge. FIG. 2C shows the forskolin-induced short-circuit current response of jejunum segments from Vehicle- and OVA-treated mice following exposure to the ion channel blockers DIDS (100 μM) or CFTRInh172 (20 μM) in the mucosal reservoir inside a Ussing chambers system. Transepithelial resistance (TER) and FITC-dextran flux of jejunum segments (FIG. 2D and FIG. 2E, respectively) are shown from Vehicle- and OVA-treated BALB/c WT mice within 60 minutes of the 7th OVA challenge. OVA-treated mice were sensitized with OVA-alum and received seven o.g. OVA challenges. Vehicle-treated mice are OVA-sensitized mice that were challenged with vehicle (saline) and did not develop anaphylaxis symptoms. Data are represented as the mean±SD; n=3-7 mice per group from 2-3 representative experiments. **** P<0.0001, ***P<0.001, ***P<0.01, **P<0.05, ns>0.05.

FIGS. 3A-3C are images of Western blot analyses illustrating that antigen challenge induced paracellular dysfunction that was associated with degradation of adherence and tight junction proteins. FIG. 3A is an image of Western blot protein analysis of Claudin-1, Claudin-2, Claudin-3, Claudia-5, Occludin, and Keratin-8. FIG. 3B is an image of Western blot protein analysis of E-cadherin, Dsg-2, and Dsc-2. FIG. 3C is an image of Western blot protein analysis of Dasg-2. Protein was extracted from mice from jejunal epithelial cells isolated pre-6th or 30 minutes post-the 7th oral antigen challenge, Actin and GAPDH were used as a loading control. MW, Molecular weight. Each column represents a single mouse, FIG. 3D includes images of immunofluorescence analysis of E-cadherin, Claudia-1, and Claudia-2 (white) from isolated intestinal epithelial cells. Nuclei were visualized with DAPI (blue). Small intestines were harvested from allergic mice 30 minutes following the 7th oral antigen challenge.

FIGS. 4A-4C are graphs illustrating that allergen exposure leads to temporal loss of epithelial transcellular and paracellular dysfunction. FIG. 4A shows Isc baseline and FIG. 4B shows TER following the 6th challenge. Allergic mice were left to recover following the 6^{th} challenge, then jejunal segments were removed and placed in the Ussing chambers and exposed to OVA into the apical side of the Ussing chambers system. FIGS. 4C and 4D, respectively, show Isc and TER of jejunum samples obtained every 24 hours after 6th OVA oral gavage or Vehicle. Data are represented as the mean±SD, n=3 mice per group. ****P<0.0001, ***P<0.001, **P<0.01, *P<0.05, ns>0.05.

FIGS. 5A and 5B are graphs showing Isc baseline response and TER, respectively, and demonstrate that altered SI transcellular and paracellular permeability is required for the development of the food-induced symptom of secretory diarrhea. Following the 6th challenge, naive or sensitized mice were grouped according to secretory diarrhea development. Mice demonstrating profuse liquid stool were recorded as diarrhea-positive. Data are represented as the mean±SD, n=4-7 mice per group. ****P<0.0001, ***P<0.001, **P<0.01, *P<0.05, ns=0.05.

FIG. 6 includes diagrams and graphs illustrating that in vivo treatment with chloride channel blocker (GlyH101) and GlyH101 plus protease inhibitors (AEBSF) attenuated the effect of oral antigen challenge on electrophysiological parameters and paracellular leakage. FIG. 6A is a schematic diagram showing the experimental regimen, FIGS. 6B and 6C are graphs showing the Isc baseline and forskolin-induced responses (ΔIsc), respectively. FIGS. 6D and 6E are graphs showing the TER and FITC-dextran flux responses, respectively of jejunal segments from OVA-sensitized and oral challenged mice (7th challenge) following pretreatment with GlyH101 and protease inhibitor (AEBSF) alone or in combination. OVA-sensitized mice received repeated OVA challenge (six challenges) and mice that demonstrated evidence of food allergy were stratified into indicated groups. Mice received either 0.5 mM GlyH101 (oral gavage) 15 min prior to the 7th OVA-challenge or 500 μg AEBSF (i.v.) 2 h prior to 7th OVA-challenge, alone or in combination and subsequently received oral gavage (OVA). Following the 60 minute observational period, jejunal segments were removed and mounted in a Ussing chamber system and physiological measurements were recorded as described in the Examples. Vehicle represents unsensitized mice that received vehicle oral gavage challenge. Data are represented as the mean±SD, n=3-7 mice per group. ****P<0.0001, ***P<0.01, **p<0.01, *P<0.05, ns>0.05.

FIG. 7 is a graph illustrating that in vivo treatment with chloride channel blocker (GlyH101) and protease inhibitors (AEBSF) attenuated the symptoms of food-induced anaphylaxis. The graph shows chi-square analysis of mice with or without secretory diarrhea. Following the 6th OVA-challenge, allergic mice were stratified into two groups (Vehicle or GlyH101+AEBSF group). 0.5 mM GlyH101 was given orally 15 minutes before the 7th OVA-challenge. 500 μg, AEBSF was given i.v 2 hours before the challenge.

### DETAILED DESCRIPTION OF THE INVENTION

The present disclosure is predicated, at least in part, on the discovery that fragments of adherence junction proteins, such as the desmoglein-2 (SSG-2) protein, are present in small intestine lysates from mouse models of food allergy. Assays for detecting such protein fragments may be used to identify food allergic reactions in humans,

### Definitions

To facilitate an understanding of the present technology, a number of terms and phrases are defined below. Additional definitions are set forth throughout the detailed description.

The term “allergy,” as used herein, refers to a chronic condition involving an abnormal or pathological immune reaction to a substance (i.e., an “allergen”) that is ordinarily harmless in normal/healthy individuals. An “allergen” refers to any substance (e.g., an antigen) that induces an allergic reaction in a subject. Examples of allergens include, but are not limited to, aeroallergens (e.g., dust mite. mold, spores, plant pollens such as tree, weed, and grass pollens), food products (milk, egg, soy, wheat, nut, or fish proteins), animal products (e.g., cat or dog hair), drugs (e.g., penicillin), insect venom, and latex.

As used herein, the terms “nucleic acid,” “polynucleotide,” “nucleotide sequence,” and “oligonucleotide” are used interchangeably and refer to a polymer or oligomer of pyrimidine and/or purine bases, preferably cytosine, thymine, and uracil, and adenine and guanine, respectively (See Albert. L. Lehninger, Principles of Biochemistry, at 793-800 (Worth Pub. 1982)). The terms encompass any deoxyribonucleotide, ribonucleotide, or peptide nucleic acid component, and any chemical variants thereof, such as methylated, hydroxymethylated, or glycosylated forms of these bases. The polymers or oligomers may be heterogenous or homogenous in composition, may be isolated from naturally occurring sources, or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002) and U.S. Pat. No. 5,034,506), locked nucleic acid (LNA; see Wahlestedt et al., Proc. Natl. Acad. U.S.A., 97: 5633-5638 (2000)), cyclohexenyl nucleic acids (see Wang, J. Am. Chem. Soc., 122: 8595-8602 (2000)), and/or a ribozyme. The terms “nucleic acid” and “nucleic acid sequence” may also encompass a chain comprising non-natural nucleotides, modified nucleotides, and/or non-nucleotide building blocks that can exhibit the same function as natural nucleotides (e.g., “nucleotide analogs”).

The terms “peptide,” “polypeptide,” and “protein” are used interchangeably herein, and refer to a polymeric form of amino acids comprising at least two or more contiguous amino acids, which can include coded and non-coded amino acids, chemically or biochemically modified or derivatized amino acids, and polypeptides having modified peptide backbones.

As used herein, the terms “treatment,” “treating,” and the like refer to obtaining a desired pharmacologic and/or physiologic effect. Preferably, the effect is therapeutic, i.e., the effect partially or completely alleviates or cures an injury, disease, and/or an adverse symptom attributable to the injury or disease. Similarly, a “therapeutic agent,” is any substance, molecule, or compound that is capable of alleviating or curing an injury, disease, and/or adverse symptom when administered to a subject in need thereof. To this end, the methods described herein desirably comprise administering a “therapeutically effective amount” of a therapeutic agent. A “therapeutically effective amount” refers to an amount effective, at dosages and for periods of time necessary, to achieve a desired therapeutic result. The therapeutically effective amount may vary according to factors such as the injury severity, age, sex, and weight of the individual, and the ability of therapeutic agent to elicit a desired response in the individual.

As used herein, the terms “immunogen” and “antigen” refer to an agent (e.g., an allergen or a microorganism (e.g., bacterium, virus or fungus)) and/or portion or component thereof that is capable of eliciting an immune response in a subject.

The term “immunoglobulin” or “antibody,” as used herein, refers to a protein that is found in blood or other bodily fluids of vertebrates, which is used by the immune system to identify and neutralize foreign objects, such as bacteria and viruses. Typically, an immunoglobulin or antibody is a protein that comprises at least one complementarity determining region (CDR). The CDRs form the “hypervariable region” of an antibody, which is responsible for antigen binding. A whole immunoglobulin typically consists of four polypeptides: two identical copies of a heavy (H) chain polypeptide and two identical copies of a. light (L) chain polypeptide. Each of the heavy chains contains one N-terminal variable (V_{H}) region and three C-terminal constant (C_{H}1, C_{H}2, and C_{H}3) regions, and each light chain contains one N-terminal variable (V_{L}) region and one C-terminal constant (C_{L}) region. The light chains of antibodies can be assigned to one of two distinct types, either kappa (κ) or lambda (λ), based upon the amino acid sequences of their constant domains. In a typical antibody, each light chain is linked to a heavy chain by disulphide bonds, and the two heavy chains are linked to each other by disulphide bonds. The light chain variable region is aligned with the variable region of the heavy chain, and the light chain constant region is aligned with the first constant region of the heavy chain. The remaining constant regions of the heap^{,} chains are aligned with each other.

The term “monoclonal antibody,” as used herein, refers to an antibody produced by a single clone of B lymphocytes that is directed against a single epitope on an antigen. Monoclonal antibodies typically are produced using hybridoma technology, as first described in Köhler and Milstein, Eur. J. Immunol., 5: 511-519 (1976). Monoclonal antibodies may also be produced using recombinant DNA methods (see, e.g., U.S. Pat. No. 4,816,567), isolated from phage display antibody libraries (see, e.g., Clackson et al. Nature, 352: 624-628 (1991)); and Marks et al.. J. Biol., 222: 581-597 (1991)), or produced from transgenic mice carrying a fully human immunoglobulin system (see, e.g., Lonberg, Nat. Biotechnol., 23(9): 1117-25 (2005), and Lonberg, Handb. Exp. Pharmacol., 181: 69-97 (2008)). In contrast, “polyclonal” antibodies are antibodies that are secreted by different B cell lineages within an animal. Polyclonal antibodies are a collection of immunoglobulin molecules that recognize multiple epitopes on the same antigen.

The terms “fragment of an antibody,” “antibody fragment,” and “antigen-binding fragment” of an antibody are used interchangeably herein to refer to one or more fragments of an antibody that retain the ability to specifically bind to an antigen (see, generally, Holliger et al., Nat. Biotech., 23(9): 1126-1129 (2005)). An antibody fragment desirably comprises, for example, one or more CDRs, the variable region (or portions thereof), the constant region (or portions thereof), or combinations thereof Examples of antibody fragments include, but are not limited to, (i) a Fab fragment, which is a monovalent fragment consisting of the V_{L}, V_{H}, C_{L}, and C_{H1} domains, (ii) a F(ab′)₂ fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)₂ fragment using mild reducing conditions, (v) a disulfide-stabilized Fv fragment (dsFv), and (vi) a domain antibody (dAb), which is an antibody single variable region domain (V_{H} or V_{L}) polypeptide that specifically binds antigen.

The terms “host” or “subject.” as used herein, refer to an individual to be treated by (e.g,, administered) the compositions and methods of the present invention. Subjects include, but are not limited to, mammals (e.g., murines, simians, equines, bovines, porcines, canines, felines, etc.), and most preferably includes humans. In the context of the invention, the term “subject” generally refers to an individual who is suspected of suffering from, or diagnosed as suffering from, a food allergy.

A used herein, the term “immune response” refers to a response by the immune system of a subject. For example, immune responses include, but are not limited to, a detectable alteration (e.g., increase) in Toll-like receptor (TLR activation, lymphokine (e.g., cytokine (e.g., Th1 or Th2 type cytokines) or chemokine) expression and/or secretion, macrophage activation, dendritic cell activation, T cell activation (e.g., CD4+ or CD8+T cells), NK cell activation, and/or B cell activation (e.g., antibody generation and/or secretion). Additional examples of immune responses include binding of an immunogen (e.g., antigen) to an MHC molecule and inducing a cytotoxic T lymphocyte (“CTL”) response, inducing a B cell response (e.g., antibody production), and/or T-helper lymphocyte response, and/or a delayed type hypersensitivity (DTH) response against the antigen from which the immunogenic polypeptide is derived, expansion (e.g., growth of a population of cells) of cells of the immune system (e.g., cells, B cells (e.g., of any stage of development (e.g., plasma cells), and increased processing and presentation of antigen by antigen presenting cells. An immune response may be to immunogens that the subject's immune system recognizes as foreign (e.g., non-self antigens from microorganisms (e.g., pathogens), or self-antigens recognized as foreign). Thus, it is to be understood that, as used herein, “immune response” refers to any type of immune response, including, but not limited to, innate immune responses (e.g., activation of Toll receptor signaling cascade) cell-mediated immune responses (e.g., responses mediated by T cells (e.g., antigen-specific T cells) and non-specific cells of the immune system), and humoral immune responses (e.g., responses mediated by B cells (e.g., via generation and secretion of antibodies into the plasma, lymph, and/or tissue fluids). The term “immune response” is meant to encompass all aspects of the capability of a subject's immune system to respond to antigens and/or immunogens (e.g., both the initial response to an immunogen (e.g., a pathogen) as well as acquired (e.g., memory) responses that are a result of an adaptive immune response).

### Determining the Presence of DSG-2 Protein Fragments

In some embodiments, the disclosure provides a method comprising: (a) obtaining a sample from a subject suspected of having a food allergy, and (b) determining the presence of one or more fragments of the desmoglein-2 (DSG-2) protein using an immunoassay. Any suitable sample type, as described herein, may be obtained from any subject suspected of having a food allergy: A subject (e.g., a human) is “suspected of having a food allergy” if the subject is predisposed to experiencing a food allergy. This predisposition may be genetic (e.g., a particular genetic tendency to experience the food allergy), or due to other factors (e.g., environmental conditions, exposures to immunogenic compounds present in certain foods, etc.). Thus, the present invention is not to be limited to any particular risk (e.g., any human may be susceptible to experiencing a food allergy), nor is the present invention limited to any particular food allergy.

The disclosed method may be used to determine the presence of any protein associated with a cell-cell junction. The terms “cell-cell junction,” “cell junction,” and “cell-to-cell junction,” are used interchangeably herein to refer to specialized regions of connection between two cells or between a cell and the extracellular matrix. Cell junctions generally can be classified into three categories: occluding junctions, anchoring junctions, and communicating junctions. Occluding junctions, also known as “tight junctions,” seal cells together in an epithelium in a way that prevents even small molecules from leaking from one side of the sheet to the other. Tight junctions are composed of a branching network of sealing strands, with each strand acting independently from the others. Each strand is formed from a row of transmembrane proteins embedded in both plasma membranes, with extracellular domains joining one another directly. There are at least 40 different proteins composing the tight junctions, which include both transmembrane and cytoplasmic proteins (see, e.g., Itallie et al., Cold Spring Harbor Perspectives in Biology, 1 (2): a002584 (2009)). The three major transmembrane proteins are occludin, claudins, and junction adhesion molecule (JAM) proteins. Anchoring junctions mechanically attach cells (and their cytoskeletons) to their neighbors or to the extracellular matrix. Anchoring junctions hold cells together and include, for example, adherence junctions and desmosomes. Anchoring junctions are formed by transmembrane adhesion proteins that belong to the cadherin family, and focal adhesions and hemidesmosomes which bind cells to the extracellular matrix and are formed by transmembrane adhesion proteins of the integrin family. Communicating junctions mediate the passage of chemical or electrical signals from one interacting cell to its partner, and include, for example, gap junctions and chemical synapses. Cell junctions are further described in, e.g., Alberts et al, (eds), Molecular Biology of the Cell. 4th edition, New York: Garland Science (2002).

In certain embodiments, the disclosed method comprises determining the presence of one or more desmosome protein fragments, such as one or more fragments of the desmoglein-2 (DSG-2) protein. Desmosomes are intercellular junctions that tether intermediate filaments to the plasma membrane. Desinogleins and desmocollins are members of the cadherin protein superfamily and are transmembrane proteins that mediate adhesion at desmosomes. The extracellular domains of the desmogleins and desmocollins mediate adhesion, whereas the cytoplasmic tails associate with the desmosomal plaque proteins. The outer dense plaque consists of the cytoplasmic tails of the desmosomal cadherins, which bind to members of the armadillo and plakin family of linker proteins (Kowalczyk et al., Biophys Chem., 50: 97-112 (1994); Getsios et al., Nat Rev Mol Cell Biol, 5: 271-281 (2004); and Garrod and Chidgey, Bloch. Biophys. Acta, 1778: 572-587 (2008)).

Desmoglein-2 is a 122.2 kDa protein composed of 1118 amino acids. Destnoglein-2 is a calcium-binding transmembrane glycoprotein component of desmosomes in vertebrate cells. The gene encoding desmoglein-2, Dsg-2, is expressed in desmosome-containing tissues, such as cardiac muscle, colon, colon carcinoma, and other simple and stratified epithelial-derived cell lines (see, e.g., Koch et al., J. Cell Biol., 55: 200-208, 1991; and Arnemann et al., Genomics, 13: 484-486 (1992)). Desmoglein-2 is the only desmoglein isoform expressed in cardiomyocytes. The nucleic acid and amino acid sequences of DSC-2 are publicly available from the National Center for Biotechnology under Accession Nos. NM_901943.5, NP_001934.2, and NG_007072.3.

The disclosed method may be used to determine the presence of any known or as yet unidentified DSC-2 protein fragment, Isoforms of the desmosomal cadherins are expressed in a tissue-specific and differentiation-specific pattern (Dusek et al. J Dermatol Sci., 45: 7-21 (2007); and Mahoney et al., Exp Dermatol., 15: 101-109 (2006)). While all isoforms (i.e., Dsg 1-4 and Dsc 1-3) are expressed in the epidermis, only Dsg-2 and Dsc-2 are expressed in cardiac myocytes and in the intestinal epithelium. In addition, the extracellular and intracellular domains of the Dsgs have been shown to be targeted by matrix tnetalloproteinases and cysteine proteases, respectively, and proteolysis may be a physiologic and/or pathologic mechanism by which desmosomal adhesion is regulated (see, Kolegrafi et al., Cell Adh Migr., 5(4): 306-314 (2011); Ramani et al., i BMC Cancer, 8: 373 (2008); Nava et al., Mol. Biol Cell., 18: 4565-4578 (2007); Cirillo et al., J Cell Biochem., 103: 598-606 (2008); Borgono et al., J Biol Chem., 282: 3640-3652 (2007); Jiang et al., J Biol Chem., 286: 9127-9135 (2011); Amagai et al., Nat Med., 6: 1275-1277 (2000); Dusek et al., J Biol Chem., 281: 3614-3624 (2006); and Bech-Serra et al., Mol Cell Biol., 26: 5086-5095 (2006)). For example, the pro-inflammatory cytokines interferon-gamma (IFN-γ) and tumor necrosis factor-alpha (TNF-α) have been shown to induce DSC-2 intracellular cleavage and generation of a ˜55 kDa fragment (Yulis et al., Cell Death Dis, 9: 389 (2018)), whith is mediated by caspase-8. Other pro-inflammatory mediators induce proteolytic cadherin cleavage during mucosal inflammation (see, e.g., Kamekura et al., Mol. Biol. Cell, 26: 3165-43177 (2015); and Nava et al., Tissue Barriers, 1: e24783 (2013)). Further studies have demonstrated that DSG cleavage fragments, as opposed to the full-length protein, actively regulate cellular processes, including apoptosis and differentiation (Nava et al., supra; and Getsios et al., J Cell Biol., 185: 1243-1258 (2009)). In some embodiments, the method comprises determining the presence of a 22 kDa DSG-2 protein fragment, a 30 kDa DSG-2 protein fragment, and/or a 75 kDa DSG-2 protein fragment.

A “fragment” of a protein or polypeptide desirably comprises at least 3 consecutive amino acid residues (e.g., about 3 to about 1,200 amino acids). In some embodiments, a “fragment” of a protein or polypeptide comprises 3 or more (e.g., 5 or more, 10 or more, 15 or more, 20 or more, 25 or more, 30 or more, 40 or more, or 50 or more) amino acids, but less than 1,200 (e.g., 1,000 or less, 800 or less, 700 or less, 600 or less, 500 or less, 400 or less, 300 or less, 200 or less, or 100 or less) amino acids. In other embodiments, a portion of an amino acid sequence is about 3 to about 500 amino acids (e.g,, about 10, 100, 200, 300, 400, or 500 amino acids), about 3 to about 300 amino acids (e.g., about 20, 50, 7:5, 95, 150, 175, or 200 amino acids), or about 3 to about 100 amino acids (e.g., about 15, 25, 3:5, 40, 45, 60, 65, 70, 80, 85, 90, 95, or 99 amino acids), or a range defined by any two of the foregoing values.

The presence of the one or more DSG-2 protein fragments may be determined or detected using any suitable type of immunoassay. The term “immunoassay,” as used herein, refers to a biochemical test that measures the presence or concentration of a macromolecule or a, small molecule in a solution through the use of an antibody or an antigen. The molecule detected by the immunoassay is often referred to as an “analyte” and is in many cases a protein. In the context of the present disclosure, the presence or amount of the one or more DSG-2 protein fragments can be determined using antibodies and detecting specific binding to the one or more DSG-2 protein fragments present in the sample. For example, an antibody, or antibody fragment thereof, may specifically bind to at least one DSG-2 protein fragment. If desired, one or more of the antibodies can be used in combination with one or more commercially available monoclonal/polyclonal antibodies. Such antibodies are available from companies such as R&D Systems, Inc. (Minneapolis, Minn.) and Enzo Life Sciences International, Inc. (Plymouth Meeting, Pa.).

Any suitable type of immunoassa.y may be used to determine the presence or amount of the one or more DSG-2 protein fragments in a sample. Examples of suitable immunoassay systems and formats are known in the art and include Western blot, immunofluorescence microscopy, sandwich immunoassay (e.g., radioisotope detection (radioimmunoassay (RIA)) and enzyme detection (enzyme immunoassay (EIA) or enzyme-linked immunosorbent assay (ELISA) (e.g., QUANTIKINE™ ELISA assays, R&D Systems, Minneapolis, Minn.)), fluoroimmunoassa.y (FIA), chemiluminescent immunoassay (CLIA), counting immunoassay (CIA), competitive inhibition immunoassay (e.g., forward and reverse), enzyme multiplied. immunoassay technique (EMIT), a competitive binding assay, bioluminescence resonance energy transfer (BRET), one-step antibody detection assay, capture on the fly assay, lateral flow assay, single molecule detection assay, etc. Immunoassay methods and formats are further described in, e.g., Wild, D. (ed.), The Immunoassay Handbook: Theory and Applications of Ligand Binding, ELISA and Related Techniques 4th Edition, Elsevier Science (2013). Other methods of detection include those described in, for example, International Patent Application Publications WO 2016/161402 and WO 2016/161400; and Adamczyk et al., Anal, Chim. Acta, 579(1): 61-67 (2006). Specific immunological binding of an antibody to a specific DSG-2 protein fragment can be detected via direct labels, such as fluorescent or luminescent tags, metals and radionuclides attached to the antibody, or via indirect labels, such as alkaline phosphatase or horseradish peroxidase.

A homogeneous or heterogeneous immunoassay format may be used, In this regard, measurement of an analyte (e.g., an allergen) requires a means for distinguishing the bound fraction (antigen-antibody complex) from the unbound fraction (free antigen or free antibody depending on the type of immunoassay). To achieve this, heterogeneous immunoassays require a physical separation step while homogeneous immunoassays do not. After the two fractions can be distinguished, detection of the label in the appropriate fraction can occur.

In some embodiments, the disclosed method comprises determining the presence of additional cell junction proteins, or fragments thereof, in addition to the one or more DSG-2 protein fragments. For example, the method may further comprise determining the presence of one or more fragments of an adherent junction (AJ) protein in the sample. Adherent junction proteins include, but are not limited to, cadherins, p120, γ-catenin (pla.koglobin), and α-catenin. The cadherins are a family of transmembrane proteins that form homodimers in a calcium-dependent manner with other cadherin molecules on adjacent cells. Examples of cadherin proteins include classical cadherins, such as epithelial (E) cadherin, neuronal (N) cadherin, and vascular epithelium (VE) cadherin (Halbleib, J. M. and W. J. Nelson, Genes & Dev., 20: 3199-3214 (2006)). p120 binds the juxtametnbrane region of the cadherin, β-catenin binds the catenin-binding region of the cadherin, and a-catenin binds the cadherin indirectly via β-catenin or plakoglobin and links the actin cytoskeleton with cadherin (Ferreri D. M., Vincent P. A., “Signaling to and through the Endothelial Adherens Junction,” in Laflamme S. E., Kowalczyk A. P. (eds.)., Cell Junctions: Adhesion, Development, and Disease, Wiley VCH (2008)). For example, the method may further comprise determining the presence of one or more fragments of E-cadherin protein in the sample. The presence of adherent junction proteins in the sample may be determined using any of the methods described herein for determining the presence of one or more USG-2 protein fragments.

### Methods of Treating Food Allergy

The present disclosure also provides method of treating a food allergy in a subject in need thereof, which method comprises: (a) determining the presence of one or more fragments of the desmoglein-2 (DSG-2) protein in a sample obtained from the subject using an immunoassay; wherein the presence of one or more fragments of the USG-2 protein indicates that the subject has a food allergy; and (b) administering a therapeutic agent to the subject, whereby the food allergy is treated. Descriptions of the subject, one of more DSG-2 protein fragments, immunoassay, and components thereof set forth above also apply to those same aspects of the aforementioned method of treating food allergy.

It will be appreciated that food allergies are atopic disorders that are mechanistically distinct from non-atopic disorders, such as celiac disease. Food allergies can be broadly classified into those that are IgE-mediated, those that are mediated by both ligE-dependent and IgE-independent pathways (mixed), and those that are not IgE-mediated. The subject to be treated may be suffering from an allergy to any one or combination of food allergens. For example, the food allergy may be a peanut allergy, a tree nut allergy, a dairy allergy, a wheat allergy, a soy allergy, an egg allergy, a shellfish allergy, a meat allergy, and/or a corn allergy. The immune mechanisms underlying food allergies are further described in, e.g., Wong et al., Nat Rev Immunol., 16(12): 751-765 (2016).

The presence of one or more DSG-2 protein fragments in the sample indicates that the subject has a food allergy; and, in such cases, the method comprises administering a therapeutic agent to the subject. Currently, there is no definitive treatment for food allergy, and the standard of care includes avoidance of food allergens and treatment of the symptoms of food allergy (Boyce et al. J. Immunol., 126: S1-58 (2010)). As such, the therapeutic agent may be any agent that ameliorates symptoms of food allergy. Such agents include, but are not limited to, epinephrine (also known as adrenaline) and antihistamines. Adrenaline can reverse edema, urticaria, bronchospasm, hypotension, and gastrointestinal symptoms within minutes. In addition, early treatment with adrenaline after allergen exposure (such as within the first six minutes after exposure) is more effective than later treatment (such as more than twenty minutes after the onset of reaction), and early response is a crucial factor in preventing death from anaphylaxis (Ho et al,, Clin Rev. Allergy 46: 225-240 (2014)). Any suitable antihistamine may be administered to the subject, depending on the particular symptom or symptoms experienced. For example, diphenhydramine (BENADRYL®), or the more specific H1 receptor blockers such as cetirizine (ZYRTEC®), may be used to treat localized food allergy symptoms (e.g,, itching, sneezing, hives, and rashes). In other embodiments, gastrointestinal symptoms can be treated with H2 receptor blockers, such as famotidine.

Recent progress has been observed using immunotherapy to desensitize individuals to potential food allergens. Thus, the therapeutic agent may also be one or more immunotherapeutie agents or treatment regimens. In some embodiments, the immunotherapy involves antigen desensitization. Desensitizing immunotherapy is generally delivered sublingually, orally, or through the skin. Sublingual immunotherapy, or SLIT, involves administering a liquid extract of the allergen under the tongue, where it is held for several minutes. Daily allergen doses begin in the submilligram range and increase gradually over a period of days or weeks. The first double-blind, placebo-controlled trial of SLIT for food allergy was published in 2005 (Enrique et al., J. Allergy Clin. Immunol., 116: 1073-1079 (2005)), and a large, multicenter, randomized, placebo-controlled, double-blind, crossover study in 2013 evaluated SLIT for peanut allergy (Fleischer et al., J. Allergy Clin. Immunol., 131: 119-127 (2013)). In oral immunotherapy, or OIT, a low dose of allergen (in the milligram range) is ingested daily and the dose is gradually increased (e.g., every two weeks) over a period of several months. Because of the larger allergen doses that are used in OIT compared with other forms of immunotherapy, patients can often be desensitized not only to amounts of the allergen sufficient to avoid a life-threatening reaction due to accidental exposure, but also to the extent that they are able to consume gram amounts of allergenic foods. Epicutaneous immunotherapy, or EPIT, employs an adhesive containing microgram amounts of allergen to deliver antigen to the skin surface. This route of delivery seems to have fewer and less intense side effects than Off, and some subjects may prefer wearing a skin patch to orally consuming the same food allergen each day.

In other embodiments, the therapeutic agent may comprise one or more monoclonal antibodies. Several monoclonal antibodies have been developed to block the processes associated with allergic immune responses. For example, the monoclonal antibody omalizumab (XOLAIR®) binds to the Fe region of IgE antibodies, blocking IgE binding to FcεRI and thus preventing the Fc receptor-mediated activation and degranulation of mast cells and basophils (Pennington et al., Nat Commun., 7: 11610 (2016)). Omalizumab was originally approved for the treatment of allergic asthma, but has been tested in combination with OTT for the treatment of food allergies in a series of smaller studies (Nadeau et al., Clin. Immunol., 127: 1622-1624 (2011); Schneider et al., J. Allergy Clin. Immunol, 132: 1365-1374 (2013); Wood et al., J. Allergy Immunol., 137: 1103-1110 (2016); and Begin et al., Allergy Asthma Clin. Immunol., 10: 7 (2014)). Monoclonal antibodies that target upstream mediators of food allergy may also be used in the methods described herein. For example, monoclonal antibodies that bind to IL-5, such as mepolizumab (NUCALA®) and reslizumab (CINQAIR®), have been evaluated for treating eosinophilic oesophagitis (EoE), which can be triggered by milk allergens (Assa'ad et al., Gastroenterology 141: 1593-1604 (2011); and Spergel et al., J. Allergy Clin. Immunol., 129: 456-463 (2012)). Therapeutic monoclonal antibodies may be administered in conjunction with immunotherapies as described above. Current and future potential treatments for food allergies are described in detail in, for example, Yu et al., Nat Rev Immunol., 16(12): 751-765 (2016).

### Kits

Also provided herein is a kit for use in performing the above-described methods. The kit may include reagents for determining the presence of the one or more DS(3-2 protein fragments, and optionally reagents for determining the presence of other adherent junction (AJ) proteins in a sample. Such reagents include, for example, monoclonal antibodies and labeling reagents.

Ideally, the kit also comprises instructions for carrying out the methods described herein. instructions included in the kit may be affixed to packaging material or may be included as a package insert. The instructions may be written or printed materials, but are not limited to such. Any medium capable of storing such instructions and communicating them to an end user is contemplated by this disclosure. Such media include, but are not limited to, electronic storage media (e.g., magnetic discs, tapes, cartridges, chips), optical media (e,g., CD ROM), and the like. The term “instructions” may also include the address of an intern.et site that provides the instructions.

The kit may further comprise reference standards for quantifying the one or more protein fragments. The reference standards may be employed to establish standard curves for interpolation and/or extrapolation of the protein fragment concentrations. The kit may include reference standards that vary in terms of concentration level. For example, the kit may include one or more reference standards with either a high concentration level, a medium concentration level, or a low concentration level. Ranges of concentrations for the reference standard can be optimized per the assay.

The kit may also include quality control components (for example, sensitivity panels, calibrators, and positive controls). Preparation of quality control reagents is well-known in the art and is described on insert sheets for a variety of diagnostic products. Sensitivity panel members optionally are used to establish assay performance characteristics, and are useful indicators of the integrity of the kit reagents and the standardization of assays.

The kit may also optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit may additionally include one or more other controls. One or more of the components of the kit can be lyophilized, in which case the kit can further comprise reagents suitable for the reconstitution of the lyophilized components. One or more of the components may be in liquid form.

The various components of the kit optionally are provided in suitable containers as necessary. The kit further can include containers for holding or storing a sample (e.g., a container or cartridge for a urine, saliva, plasma, cerebrospinal fluid, or serum sample, or appropriate container for storing, transporting or processing tissue so as to create a tissue aspirate). Where appropriate, the kit optionally can contain reaction vessels, mixing vessels, and other components that facilitate the preparation of reagents or the sample. The kit can also include one or more sample collectionlacquisition instruments for assisting with obtaining a sample, such as various blood collection/transfer devices (e.g., microsampling devices, micro-needles, or other minimally invasive pain-free blood collection methods; blood collection tube(s); lancets; capillary blood collection tubes; other single fingertip-prick blood collection methods; buccal swabs, nasal/throat swabs; 16-gauge or other size needle, syringes, sterile container, or canula, for obtaining, storing or aspirating tissue samples).

The following examples further illustrate the invention but, of course, should not be construed as in any way limiting its scope,

### EXAMPLES

The following materials and methods were used in the experiments described in the Examples.

Animals. 6-8-week-old BALB/c wild type (WT) mice were obtained from the National Cancer institute (Bethesda, Md., USA) and bred in-house at Cincinnati Children's Hospital Medical Center (CCHMC) (Cincinnati, Ohio, USA) and at the University of Michigan (UM) (Ann Arbor, Mich., USA). Intestinal IL-9 transgenic (iIL9Tg) mice were generated as previously described (Forbes et al., Exp Med 205, 897-913 (2008)). Age-, sex-, and weight-matched littermates were used as controls in all experiments. The mice were maintained and bred in a clean barrier facility and were handled under an approved Institutional Animal Care and Use Committee protocols at CCHMC and University of Michigan animal facility.

Oral Antigen-Induced Intestinal Anaphylaxis. 4-8-week-old mice were sensitized to ovalbumin (OVA) (50 ηg of OVA/1 mg of alum in sterile saline by intraperitoneal (i.p.) injection) and received repeated oral gavage (o.g.) challenge with OVA (250 μl of OVA (50 mg) in saline or 250 μl of saline (vehicle)) as previously described (Ahrens et al., American Journal of Pathology 180, 1535-1546, doi:10.101/j.ajpath.2011.12.036 (2012)). Prior to each o.g. challenge, mice were deprived of food for 4-5 hours. Rectal temperatures were measured prior to challenge and then every 15 minutes for 60 minutes. Diarrhea was assessed by visually monitoring mice for up to 60 minutes following o.g. challenge and mice demonstrating profuse liquid stool were recorded as diarrhea-positive. Evidence of secretory diarrhea was assessed by determination of short-circuit current (Isc) of small intestine (Si) segments ex vivo in a Ussing chamber system up to 60 minutes following o.g. challenge. Mice were considered allergic if they demonstrated symptoms of anaphylaxis (hypothermia>1.5° C. Temperature loss and diarrhea) following the 6th challenge. In some experiments, mice were o.g. with 0.5 mM. N-(2-naphthalenyl)-[(3,5-dibromo-2,4-dihydroxyphenyl) methylene] glycine hydrazide (GlyH101) (EMD Millipore #219671) 15 minutes before the 7th OVA-challenge. 500 μg 4-benzenesulfonyl fluoride hydrochloride (AEBSF) (Sigma #A8456) were given intravenously (i.v.) 2 hours prior to the 7th OVA the challenge.

To track food allergen passage in the GI tract, mice were administered OVA (200 mg/ml) with 5×10⁵ FITC-labelled FLUOSPHERES™ Polystyrene Microspheres (10 μM size) (Thermo Fisher, Waltham, Mass., USA) by oral gavage and monitored for 30 minutes. The mice were euthanized, and the GI tract was surgically removed and segmented into anatomical compartments of the GI tract (stomach, duodenum, jejunum, ileum, caecum and colon). The duodenum was divided into 1.5 cm segments, jejunum into 4 cm segments, ileum into 2 cm segments, and colon into 4 cm segments. The duodenum was defined as a 3 cm GI segment distal to the pyloric sphincter. The jejunum was defined as the ˜16 cm (ii segment distal of the duodenum and 10 cm proximal from the ileocecal valve. The ileum was defined as the GI segment 10 cm proximal from the ileocecal valve. The caecum was defined as the pouch connecting to the junction of the proximal ileum and distal colon. The colon segment was ˜8 cm connecting the proximal caecum to the distal rectum. The luminal contents of the segments were flushed with phosphate-buffered saline (PBS), centrifuged, and suspended in 200 μl PBS and the fluorescence of the total contents of each segment was measured using a Bioteck multi-mode plater reader (Synergy H1) with Gen5 software.

Passive Anaphylaxis. Mice were injected i.v. with 20 μg/200 μL of anti-IgE (IgG2a mAb to mouse IgE; EM-95) and evidence of anaphylaxis was examined as previously described (Baniyash et al., Eur J immunol 14, 799-807 (1984), and Strait et al., J Allergy Clin immunol 109, 658-668. (2002)).

Solutions and drugs. The Krebs buffer used on each side of the Ussing chamber contained 4.70 mM KCl, 2.52 mM CaCl₂, 118.5 mM NaCl, 1.18 mM NaH₂PO₄, 1.64 mM MgSO₄ and 24.88 mM NaHCO₃. The tissues were allowed to equilibrate for 15 minutes in Krebs buffer containing 5.5 mM glucose. All reagents were obtained from Sigma-Aldrich (St. Louis, Mo., USA) unless stated otherwise.

Ussing chambers. 1 cm, freshly isolated, serosal-stripped segments of jejunum were mounted between the hemi-chambers of an Ussing apparatus (U2500 Dual Ussing chamber, Warner instruments, Hamden, Conn.), and 0.112 cm² of tissue was exposed to 10 ml Krebs buffer at 37° C. The transepithelial potential difference (PD) was detected with two paired electrodes that contained 4% agar in 3 M KCl. The electrodes were connected to a voltage clamp amplifier (EC-800, Epithelial voltage clamp. Warner Instruments, Hamden, Conn.), The electrode potential difference and fluid resistance were compensated before mounting tissue segments into the chamber. To establish equilibrium, PD was continuously monitored under open-circuit conditions for 15 min. Thereafter, the tissues were voltage-clamped at 0 mV while continuously measuring Isc. Voltage pulses (3-mV square waves sustained for 5 seconds) were delivered every 50 seconds to yield a current response for calculation of transepithelial resistance (TER) from Ohm's law. For ion conductance experiments, changes in Isc were determined for the cumulative addition of forskolin and acetylcholine to the serosal reservoir. After the peak response to the final concentration of each agonist was recorded, the Krebs buffer on each side of the chamber was replaced, and the tissue was allowed to equilibrate for 30 minutes. Immediately following re-equilibration, tissue was pre-incubated with ion channel blockers 4,4′-Diisothiocyanatostilbene-2,2′-disulfonate (INDS) (100 μM) or CETRInh172 (20 μM) to mucosal reservoir. Changes in Isc were measured in response to the addition of forskolin to the mucosal side. To study effects of direct allergen application, 1% OVA or equal amount saline has been directly added into apical side of the dissected jejunum mounted in the Ussing Chamber and Isc, TER were recorded as previously described (Forbes et al., supra).

Intestinal epithelial cells (IEC) preparation. A 5 cm segment of the jejunum was washed with cold PBS and 2% fetal bovine serum (FBS) and 5 mM DTT (20 min at 37° C. with shaking). Afterward, IEC were isolated by washing tissue 3 times with PBS and 2% FBS and 5 mM EDTA (10 min at 37° C. with shaking). The washing solution was then collected and. centrifuged (400 g for 10 min at 4° C.), and pellet was suspended in PBS for cell quantification and lysis. For cell lysis, isolated IEC were resuspended in R1PA buffer (0.5% Triton X-100, 0.5% NP-40, 0.5% deoxycholic acid, 0.1% SDS, 150 mM NaCl, 1 mM EGTA [pH 8.0], 1 mM EDTA, 0.2 mM sodium orthovanadate, 20 mM Tris [pH 7.4]) supplemented with protease and phosphatase inhibitors. immunoblotting was performed as previously described (Capaldo et al., Molecular Biology of the Cell 25, 2710-2719, doi.:10.1091/m.bc.E14-02-0773 (2014)).

Immunofluorescence. 5 cm segment of the jejunum was fresh frozen in O.C.T. Tissues were fixed in 95% cold ethanol for 30 minutes, followed by 1 minute of pure acetone fixation at room temperature. Primary antibody staining was performed in Hank's balanced salt solution with 3% bovine serum albumin (BSA) overnight. Secondary antibodies were incubated in 3% BSA and for 1 hour. Antibodies for Western blot were as follows: Rabbit anti-claudin-1 #51-9100 (Thermo Fisher, Waltham, Mass,, USA), rabbit anti-claudin-2 #51-6100 (Thermo Fisher, Waltham, Mass., USA), rabbit anti-claudin-3 #SAB4500434 (Sigma Aldrich, St. Louis, Mo., USA), mouse anti-dal:din-5 #35-2500 (ThermoFisher, Waltham, Mass., USA), goat anti-E-Cadherin 4AF748, goat anti-mouse JAM-A #AF1077 (R&D Systems, Minneapolis, Minn., USA), rabbit anti-cytokeratin-8 #ab53280, rabbit anti-desmoglein-2 4a.b124683 (Abcam, Cambridge, United Kingdom), mouse anti-desmocollin-2 #32-6200 (Thermo Fisher, Waltham, Mass., USA), rabbit anti-GADPH #G9545 (Sigma Aldrich, St. Louis, Mo., USA), rabbit anti-calnexin #C4731 (Sigma Aldrich, St. Louis, Mo., USA). Antibodies for immunofluorescence were as follows: rat anti-E-cadherin #53-3249-82, rabbit anti-claudin-1 #51-9000, rabbit anti-.claudin-2 #516100 (Thermo Fisher, Waltham, Mass,, USA). Nuclei were detected with DAPI. Confocal microscopy was performed using a Leica SP5 inverted microscope (Wetzlar, Gertnany) and Leica SP5 software.

Statistical analysis. Data are expressed as mean±standard deviation (SD), unless otherwise stated. Statistical significance comparing different sets of mice was determined by Student's t test. In experiments comparing multiple experimental groups, statistical differences between groups were analyzed using the one-way, nonparametric ANOVA and a Bonferroni post-test. P<0.05 was considered significant. All analyses were performed using Prism 7.0 software GraphPad. Software Inc., San Diego, Calif., USA).

### Example 1

This example demonstrates that food antigen exposure is restricted to the small intestine (SI) during a food-induced anaphylactic reaction in an animal model.

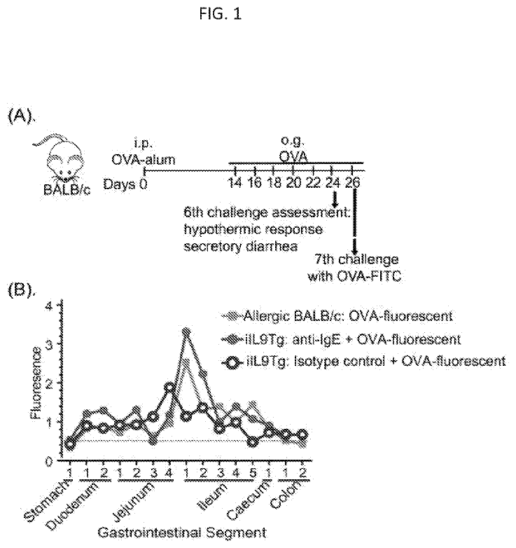

Previous work has shown that within 30 minutes of the 7th oral gavage challenge of ovalbumin (OVA) in OVA-sensitized mice, symptoms of food-induced anaphylaxis are induced, including cutaneous, respiratory, cardiovascular and gastrointestinal involvement (Ahrens et al., supra; and Sledd et al., Immunity, Inflammation and Disease 3, 420-430, doi:10.1002/iid3.80 (2015)). To ascertain the localization of dietary antigen in the GI tract during the onset of the symptoms of food-induced anaphylaxis, mice received o.g. of fluorescent-OVA, and the transit of dietary antigen along the GI tract was monitored for 30 minutes (FIG. 1A). Dietary antigen was predominantly localized in the SI, in particularly the jejunum-ileum region, with the highest concentration localized to the proximal ileum region (FIG. 1B). Minimal evidence of dietary antigen was observed in the caecum and colon from the ileocecal junction to distal colon. OVA-sensitized and challenged mice possess a heightened GI CD4⁺ Th₂, ILC2 immune response in the SI, which can alter GI peristalsis (see, e.g., Akiho et al., Am J Physiol Ga.strointest Liver Physiol 282, G226-232 (2002); Akiho et al, World J Gastrointest Pathophysiol 2, 72-81 (2011); Akiho et al., Am J Physiol Ga.strointest Liver Physiol 288, G609-615, doi:00273.2004 [pii] 10.1152/ajpgi.00273.2004 [doi] (2005); Khan et al., Infect Immun 71, 2130-2438 (2003); and Zhao et al., J Immunol 171, 948-954 (2003)).

To determine the localization of antigen in the compartment following IgE-MC activation independent of the Type-2 immune response, a passive-oral IgE mediated model of anaphylaxis was employed using transgenic mice with intestinal mastocytosis and no Th₂ activation (iIL-9Tg) (Forbes et al., supra; and Ahrens et al., supra). Fluorescent OVA in iIL-9Tg mice 30 minutes following MC activation was similar to that observed in WT mice that experienced food-induced anaphylaxis (FIG. 1B). Furthermore, the dietary antigen was restricted to the distal jejunum and jejunoileal region in iIL-9Tg mice that received isotype control and did not experience anaphylaxis, suggesting that anaphylaxis does not significantly alter dietary antigen translocation (FIG. 1B).

The results of this example demonstrate that the eliciting dietary antigen is predominantly restricted to the murine jejunoileal region and not in the caecum or colon at the corresponding time mice experience symptoms of anaphylaxis.

### Example 2

This example demonstrates that antigen challenge stimulates intestinal epithelial CFTR-dependent Cl— transport and paracellular leak.

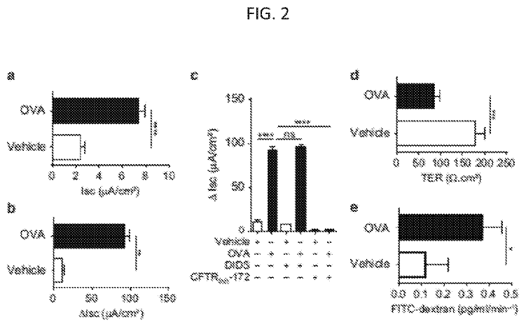

Given the observation that dietary antigen was restricted to the SI, epithelial ion transport (Short circuit current; Isc) of the SI was examined from control and food-allergic mice (within 30 minutes of food-challenge) to determine whether anaphylaxis was associated with altered intestinal epithelial permeability. Basal Isc and forskolin-induced ΔIsc of the SI of food-allergic mice was significantly increased compared to vehicle-treated mice, suggesting enhanced cAMP-dependent CF IR activity (FIGS. 2A and B). Indeed, pre-exposure of the SI epithelium to the CFTR inhibitor (CFTR^{inh172}) and not DIDS, a potent inhibitor of calcium activated Cl— transporters (Cl—/HCO₃⁻ exchanger and potassium/chloride co-transporter), abrogated the Forskolin-induced ΔIsc indicating that increased current is predominantly mediated by increased transport of Cl— via CF (FIG. 2C). Assessment of paracellular epithelial function revealed decreased Transepithelial resistance (TER), and conversely, increased macromolecular flux (FITC-Dextran flux: apical to basolateral) of the jejunum from OVA-treated mice compared to vehicle-treated mice (FIGS. 2D and 2E).

The results of this example demonstrate that dietary antigen induced a rapid SI epithelial CFTR-dependent Cl— secretory response and paracellular permeability within 30 minutes of antigen challenge.

### Exampe 3

This example demonstrates that direct exposure of OVA to jejunal SI preparations is sufficient to promote epithelial transcellular and paracellular dysfunction.

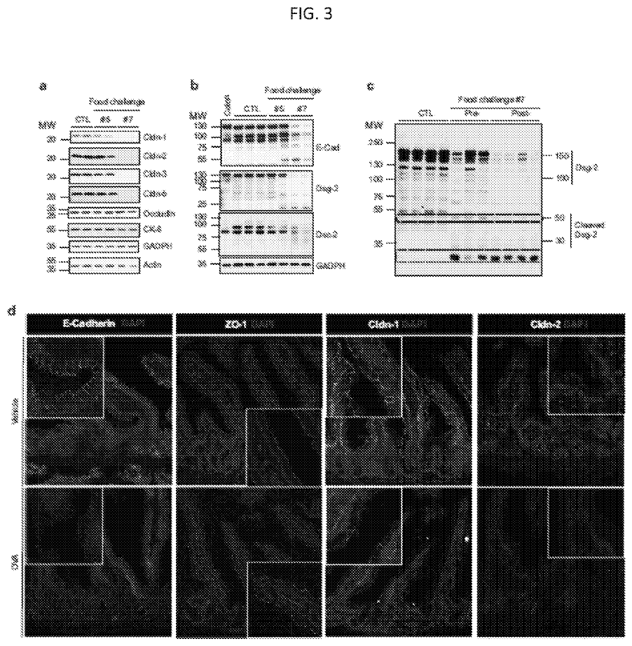

To examine whether the food-induced macromolecular leak was associated with changes in intercellular junctional proteins (JP), SI epithelial adherent junction (AJ) and tight junction (TJ) proteins were examined from naive mice and mice that demonstrated symptoms of anaphylaxis ≥1.5° C. temperature loss and diarrhea) 30 minutes following the 7th food allergen challenge. A significant reduction in the level of full length transmembrane proteins was observed in the TJ, Claudin-1, -2, -3 and -5 AJ, E-cadherin and desmoglein-2 (Dsg-2) and desmocollin-2 (Dsc-2) in SI epithelial extracts from food allergic mice compared with naive mice (FIGS. 3A and B, control (CTL) vs. food-challenge #7). Cleaved fragments of the AJ protein, E-cadherin (˜55 kDa), Dsg-2 (22 kDa), and Dsc-2 (75 kDa) also were identified in SI epithelial extracts from anaphylactic mice (FIG. 3B. food-challenge #7). The food allergic reaction was not associated with a decrease in all intestinal JP as SI TJ protein occludin was unaffected by repeated dietary food-challenge (FIG. 3A challenge 47). Keratin-8 (CK-8) immunoblotting revealed comparable levels of intestinal epithelial cells, and Actin and GAPDH staining showed similar protein loading (FIGS. 3A and B). Immunofluorescence analyses of the jejunum revealed a similar pattern of decreased levels of the TJ. Claudin-1, Claudin-2 and E-cadherin proteins in the apical junctional complex of the intestinal epithelium of allergic mice within 30 minutes of antigen challenge as compared to control mice (FIG. 3D).

To determine whether the cleaved intestinal JP in the TJ, AJ, and desmosomes (DM) was associated with development of GI symptoms of food-induced anaphylaxis, analyses were performed on mice following the fifth food-challenge that do not demonstrate symptoms of food-induced anaphylaxis following challenge (post-5th challenge). The level of TJ proteins, Claudin-1, -2, -3 and -5 were similar to those observed in naive mice (FIG. 3A; food-challenge 45). Furthermore, full-length E-cadherin, Dsg-2, and Dsc-2 were observed in SI epithelial extracts from asymptomatic mice following 5th challenge (FIG. 3B), albeit cleaved cadherin fragments (E-cadherin and Dsg-2 and Dsc-2) also were detected (FIG. 1B). Collectively these data show that cleavage of intestinal JP in the TJ, AJ and DM was associated with development of GI symptoms of food-induced anaphylaxis.

Given that cleaved fragments were detected in cadherin of AJ and DM, the Dsg-2 western blot analyses were used as a surrogate marker to determine whether a single dietary antigen-challenge induced rapid SI cadherin cleavage. To do this, Si epithelial Dsg-2 was examined in mice prior to (Pre-) and following (Post-) the 7th food-challenge. Notably, loss of the native full length Dsg-2 was observed following the 7th challenge. Also observed were decreased levels of the 50 kDa Dsg-2 cleavage fragment and accumulation of a lower molecular weight (30 kDa.) Dsg-2 fragment (FIG. 3C). These data indicate that a single allergen challenge is sufficient to induce a pronounced and rapid decrease in the full-length high molecular weight Dsg-2 protein levels and increasing low molecular weight Dsg-2 cleavage products in mice that develop food-induced anaphylaxis.

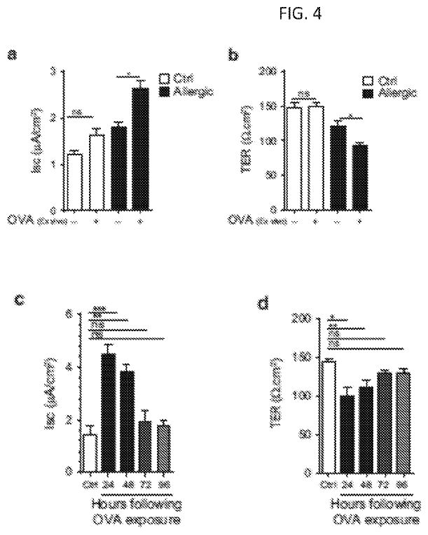

To determine whether direct antigen exposure of the SI epithelium can induce the GI epithelial dysfunction, the apical surface of SI segments from naive and food-allergic mice were exposed ex vivo to OVA in an Ussing Chamber system and assessed Isc and TER. Ex vivo exposure of OVA to the SI segment of a naive animal did not induce any significant change in Isc or TER (FIG. 4A). In contrast, OVA exposure of the allergic of SI segment stimulated an increase in Isc and a decrease in TER within 30 minutes (FIGS. 1A and 4B). The baseline Isc and TER of the SI segments from allergic mice was trending lower than that observed from naïve mice, however levels were not statistically significant (FIGS. 4A and 4B). These studies show that a single allergen challenge is sufficient to induce a pronounced and rapid decrease in intestinal epithelial barrier function that is related to enhanced ion transport and loss of intestinal epithelial barrier. To get insight into the temporal nature of the paracellular and tra.nscellular epithelial dysfunction following allergen challenge, Isc and TER of SI segments were monitored from food allergic mice following the 7th OVA-challenge. OVA-induced intestinal epithelial barrier dysfunction was maintained for at least 48 hours following dietary antigen challenge and returned to baseline levels by 72 hours (FIGS. 4C and 4D). Collectively, these studies demonstrate that dietary antigen exposure of the SI mucosal epithelium is sufficient to induce SI barrier dysfunction that can be sustained for up to 48 hours following allergen exposure.

### Exampe 4

This example describes the dissection of mechanisms of oral antigen-induced transcellular and paracellular permeability.

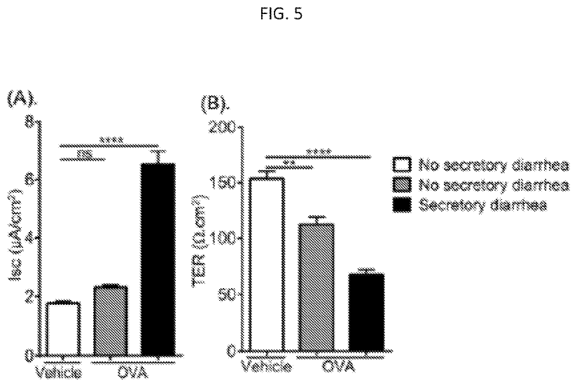

To define the relationship between SI epithelial transcellular and paracellular barrier dysfunction and the development of the dietary antigen-induced GI symptom of secretory diarrhea, the SI was observed from allergic mice that did and did not develop diarrhea following the 7th challenge. The SI segment from mice that developed secretory diarrhea within 30 minutes of food allergen-challenge had a significant increase in Isc (˜3-fold) and decreased TER (60% reduction) (FIGS. 5A and 5B). In contrast, the SI from mice that received antigen that failed to develop secretory diarrhea did not demonstrate evidence of altered Isc but did show a significant reduction in TER (˜26% reduction) (FIGS. 5A and 5B). These studies demonstrate a relationship between altered SI transcellular and paracellular permeability and the development of the food-induced symptom secretory diarrhea in food allergic mice (FIGS. 5A and B).

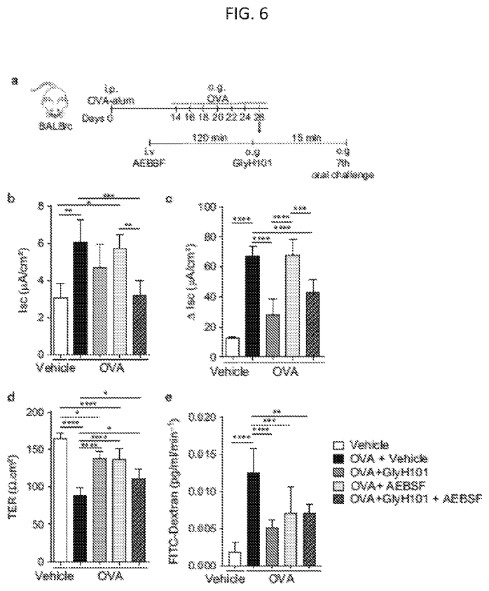

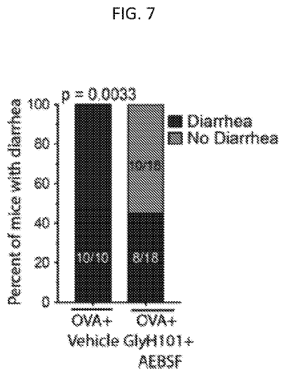

In view of the observed altered Cl- transport and the association of paracellular barrier dysfunction with JP protein degradation and dietary antigen-induced GI symptoms, it was hypothesized that the secretory diarrhea response was a consequence of CFTR-dependent Cl— secretion and proteolytic-activity. To test this hypothesis, mice that demonstrated a history of food-induced anaphylaxis (as confirmed by the 6th challenge) received a chloride channel blocker (GlyH101) (o.g 15 minutes before OVA), a protease inhibitor (AEBSF) (i.v 2 hours before OVA), or both drugs prior to the 7th challenge, and food allergen-induced SI epithelial transcellular and paracellular function was assessed (FIG. 6A). As previously shown, OVA-challenge increased basal Isc and enhanced forskolin-induced AIsc response compared to non-allergic (vehicle) mice (FIG. 2, FIG. 6B, and FIG. 6C). Pretreatment with both GlyH101 and AEBS prior to the 7th OVA-challenge reduced the Isc baseline, forskolin-induced Alsc, and TER, and conversely decreased FITC-dextran flux compared with vehicle-treated mice (FIGS. 6B and 6C). Pretreatment with GlyH101 alone dramatically inhibited forskolin-induced ΔIsc and improved the TER (FIGS. 6D and 6E). Importantly, pretreatment of mice with GlyH101 and AEBSF reduced the incidence of diarrhea in mice following the 7th OVA-challenge (FIG. 7B). 10 of 10 of food-allergic mice that received OVA developed secretory diarrhea following the 7th challenge. In contrast, only 8 of 18 mice who received GlyH101 and AEBSF developed secretory diarrhea following the 7th challenge (FIG. 7A).

The results of this example demonstrate that secretory diarrhea associated with dietary antigen-induced anaphylaxis is a consequence of food antigen-induced transcellular and paracellular SI epithelial barrier function.

### REFERENCES

The following references are herein incorporated herein in their entireties.
1. Sampson, H. A. Anaphylaxis and emergency treatment. Pediatrics 111, 1601-1608 (2003).
2. Ross, M. P. et al. Analysis of food-allergic and anaphylactic events in the national electronic injury surveillance system. J. Allergy Clin. Immunol. 121, 166-171 (2008).
3, Wang, J. & Sampson, H. A. Food anaphylaxis. Clin, Exp. Allergy 37, 651-660 (2007).
4. Sampson, H. A., Mendelson, L. & Rosen, J. P. Fatal and near-fatal anaphylactic reactions to food in children and adolescents. The New England journal of medicine 327, 380-384 (1992).
5. Schrander, J, J., Unsalan-Hooyan, R. W,, Forget, P. P. & Jansen, J. [51Cr]EDTA intestinal permeability in children with cow's milk tolerance, J. pediatr. Gastroenterol. Nutr. 10, 189-192 (1990).
6. Troncone, R., Caputo, N., Florio, G. &. Finelli, E. Increased intestinal sugar permeability after challenge in children with cow's milk allergy or intolerance. Allergy 49, 142-146 (1994).
7. Van Elburg, R., Heymans, H. S. & De, M. J. Effect of disodiumcromoglycate on intestinal permeability changes and clinical response during cows milk challenge. Source (Bibliographic Citation): Pediatr Allergy Immunol 4, 79-85 (1993).
8. Calvani, M. et al. Risk factors for severe pediatric food anaphylaxis in Italy. Pediatric allergy and immunology: official publication of the European Society of Pediatric Allergy and Immunology 22, 813-819 LID—810.1111/j.1399-3038.2011,01200.x [doi] (2011).
9. Brown, S. G. A. Clinical features and severity grading of anaphylaxis. J Allergy Clin Immunol 114, 371-376 (2004).
10. Galli, S. J. et al. Mast cells as “tunable” effector and immunoregulatory cells: recent advances. Annu. Rev. Immunol. 23, 749-786 (2005).
11. Strait, R. I., Morris, S. C.. Yang, M., Qu, X. W. & Finkelman, F. D. Pathways of anaphylaxis in the mouse. J Allergy Clin Immunol 109, 658-668. (2002).
12. Dombrowicz, D., Flamand, V., Brigman, K. K., Koller, B. H. & Kinet, J. P. Abolition of anaphylaxis by targeted disruption of the high affinity immunoglobulin E receptor alpha chain gene. Cell 75, 969-976 (1993).
13. Lorentz, A., Schwengberg, S., Mierke, C., Manns, M. P. & Bischoff, S. C. Human intestinal mast cells produce IL-5 in vitro upon IgE. receptor cross-linking and in vivo in the course of intestinal inflammatory disease. Eur J immunol 29, 1496-1503 (1999).
14. Santos, J., Benjamin, M., Yang, P. C., Prior, T. & Perdue, M. H. Chronic stress impairs rat growth and jejunal epithelial barrier function: role of mast cells. Am J Physiol Gastrointest Liver Physiol 278, G847-854 (2000).
15. Kelefiotis, D. & Vakirtzi-Lemonias, C. In vivo responses of mouse blood cells to platelet-activating factor (PAF): role of the mediators of anaphylaxis. Agents Actions 40, 150-156 (1993).
16. Strait, R. T., Morris, S. C., Smiley, K., Urban, J. F., Jr. & Finkelman, F. D. IL-4 exacerbates anaphylaxis. J Immunol 170, 3835-3842 (2003).
17. Lorentz, A., Schwengberg, S., Selige, G., Manns, Ni P. &. Bischoff, S. C. Human intestinal mast cells are capable of producing different cytokine profiles: Role of IgE receptor cross-linking and IL-4. J. Immunol. 164, 43-48 (2000).
18. Brandt, E. B. et al. Mast cells are required for experimental oral allergen-induced diarrhea. J Clin Invest 112, 1666-1677 (2003).
19. Forbes, E. E. et al. IL-9- and mast cell-mediated intestinal permeability predisposes to oral antigen hypersensitivity. J Exp Med 205, 897-913 (2008).
20. Wechsler, J. B., Schroeder, H. A., Byrne, A. J., Chien, K. B. & Bryce, P. J. Anaphylactic responses to histamine in mice utilize both histamine receptors 1 and 2. Allergy 68, 1338-1340. doi:10.1111/a11.12227 (2013).
21. Ahrens, R. et al. Intestinal mast cell levels control severity of oral antigen-induced anaphylaxis in mice. The American journal of pathology 180, 1535-1546, doi:10.1016/j.ajpath.2011.12.036 (2012).
22. Baniyash, M. & Eshhar. Z. Inhibition of IgE binding to mast cells and basophils by monoclonal antibodies to murine IgE. Eur J Immunol 14, 799-807 (1984).
23. Capaldo, C. T, et al. Proinflammatory cytokine-induced tight junction remodeling through dynamic self-assembly of claudins. Molecular biology of the cell 25, 2710-2719, doi:10.1091/mbc.E14-02-0773 (2014).
24. Sledd, J. et al. Loss of IL-4R,alpha-mediated PI3K signaling accelerates the progression of IgE/mast cell-mediated reactions. Immunity, inflammation and disease 3, 420-430, doi:10.1002/iid3.80 (2015).
25. Akiho, H., Blennerhassett, P., Deng, Y. & Collins, S. M. Role of IL-4, IL-13, and STATE in inflammation-induced hypercontractility of murine smooth muscle cells. Am J Physiol Gastrointest Liver Physiol 282, G226-232 (2002).
26. Akiho, H. , Ihara, E., Motomum, Y. & Naka.mum, K. Cytokine-induced alterations of gastrointestinal motility in gastrointestinal disorders. World J Gastrointest Pathophysiol 2, 72-81 (2011).
27. Akiho, H. et al. Interleukin-4- and -13-induced hypercontractility of human intestinal muscle cells-implication for motility changes in Crohn's disease. Am J Physiol Gastrointest Liver Physiol 288, G609-615, doi:00273.2004 [pii] 10.1152/ajpgi.00273.2004 [doi] (2005).
28. Khan. W. I. et al. Modulation of intestinal muscle contraction by interleukin-9 (IL-9) or IL-9 neutralization: correlation with worm expulsion in murine nematode infections. Infect Immun 71, 2430-2438 (2003).
29. Zhao, A. et al. Dependence of IL-4, IL-13, and nematode-induced alterations in murine small intestinal smooth muscle contractility on Stat6 and enteric nerves. J Immunol 171, 948-954 (2003).
30. Rowe, A., Jr., Rowe, A. H., Uyeyama, K. & Young, E. J. Diarrhea caused by food allergy. The Journal of allergy 27, 424-436 (1956).
31. Rowe, A. H. [Food allergy and its clinical manifestations]. Revista clinica espanola 62, 366-373 (1956).
32. Rowe, A. H. ABDOMINAL FOOD ALLERGY: ITS HISTORY, SYMPTOMATOLOGY, DIAGNOSIS AND TREATMFNT. California and western medicine 29, 317-322 (1928).
33. Simons, F. E. &. Sheikh, A. Anaphylaxis: the acute episode and beyond. BMJ (Clinical research ed.) 346, f602, doi:10.1136/bmj.f602 (2013).
34. Lieberman, P. et al. The diagnosis and management of anaphylaxis practice parameter: 2010 update. J Allergy Clin Immunol 126, 477-480 e471-442, doi:10.1016/j.jaci.2010.06.022 (2010).
35. Brown, S. G. Clinical features and severity grading of anaphylaxis. J Allergy Clin immunol 114, 371-376. doi:10.1016/j.jaci. 2004.04.029 (2004).
36. Soter, N. A., Austen, K. F. & Wasserman, S. I. Oral disodium cromoglycate in the treatment of systemic mastocytosis. The New England journal of medicine 301, 465-469, doi:10.1056/nejm197908303010903 (1979).
37. Horan, R. F., Sheffer, A. L. & Austen, K. F. Cromolyn sodium in the management of systemic mastocytosis. J Allergy Clin Immunol 85. 852-855 (1990).
38. Andre, C., Andre, F., Colin, L. & Cavagna, S. Measurement of intestinal permeability to mannitol and lactulose as a means of diagnosing food allergy and evaluating therapeutic effectiveness of disodium cromoglycate. Annals of allergy 59, 127-130 (1987).
39. Perdue, M. H., Masson, S., Wershil, B. K. & Galli, S. J. Role of mast cells in ion transport abnormalities associated with intestinal anaphylaxis. Correction of the diminished secretory response in genetically mast cell-deficient W/Wv mice by bone marrow transplantation. J Clin Invest 87, 687-693 (1991).
40. Crowe, S. E., Sestini, P. & Perdue, M. H. Allergic reactions of rat jejunal mucosa. Ion transport responses to luminal antigen and inflammatory mediators. Gastroenterology 99, 74-82 (1990).
41. Thia.garajah, J. R., Donowitz, M. & Verkman, A. S. Secretory diarrhoea: mechanisms and emerging therapies. Nature reviews. Gastroenterology & hepatology 12, 446457, doi:10.1038/nrgastro. 2015.111 (2015).
42. Barrett, K. E. & Keely, S. J. Chloride secretion by the intestinal epithelium: Molecular basis and Regulatory Aspects. Ann. Rev. Physiol. 62, 535-572 (2000).
43. Homaidan, F. R., Tripodi, J., Zhao, L. & Burakoff, R Regulation of ion transport by histamine in moue cecutn. Eur. J. Pharmacol. 331, 199-204 (1997).
44. Kim, Y. S. et al. Histamine 1 receptor-Gbetagamma-cAMP/PKA-CFTR pathway mediates the histamine-induced resetting of the supraChiasmatic circadian clock. Molecular brain 9, 49, doi:10.1186/s13041-016-0227-1 (2016).
45. Kellutn, J. M., Budhoo, M. R., Siriwardena, A. K., Smith, E. P. & Jebraili, S. A. Serotonin induces Cl— secretion in human jejunal mucosa in vitro via a nonneural pathway at a 5-HT4 receptor. The American journal of physiology 267, G357-363, doi:10.1152/ajpgi.1994.267.3.G357 (1994).
46. Collins, D., Hogan, A. M., Skelly, M. M., Baird, A. W. & Winter, D. C. Cyclic AMP-mediated chloride secretion is induced by prostaglandin F2alpha in human isolated colon. British journal of pharmacology 158, 1771-1776, doi:10.1110.1476-5381.2009.00464.x (2009).
47. Nakamura, I. et al. Mast cell-derived prostaglandin D2 attenuates anaphylactic reactions in mice. J Allergy Clin Immunol 140, 630-632.e639, doi:10.1016/j.jaci.2017.02.030 (2017).
48. Wu, D. et al. Interleukin-13 (IL-13)/IL-13 Receptor al (IL-13Rα1) Signaling Regulates intestinal Epithelial Cystic Fibrosis Transmembrane Conductance Regulator Channel-dependent Cl— Secretion*. J Biol Chem 286, 13357-13369, doi:10.1074/jbc.M110.214965 (2011).
49. Galietta, L. J. et al. IL-4 is a potent modulator of ion transport in the human bronchial epithelium in vitro. J Immunol 168, 839-845 (2002).
50. Zund, G., Madara, J. L., Dzus, A. L., Awtrey, C. S. & Colgan, S. P. Interleukin-4 and interleukin-13 differentially regulate epithelial chloride secretion. J Biol Chem 271, 7460-7464 (1996).
51. Groschwitz, K. R. &. Hogan, S. P. Intestinal barrier function: molecular regulation and disease pathogenesis. J Allergy Clin Immunol 124, 3-20; quiz 21-22, doi:S0091-6749(09)00864-1 [pii] 10.1016/j.jaci.2009,05.038 [doi] (2009).
52. Turner, J. R, Intestinal mucosal barrier function in health and disease. Nat Rev Immunol 9. 799-809, doi:nri2653 [pit] 10.1038/nri2653 [doi] (2009).
53. Turner, M. W. et al, Intestinal hypersensitivity reactions in the rat. I. Uptake of intact protein, permeability to sugars and their correlation with mucosal mast-cell activation. Immunology 63,119-124 (1988).
54. King, S. J., Miller, H. R., Newlands, G. F. & Woodbury. R. G. Depletion of mucosal mast cell protease by corticosteroids: effect on intestinal anaphylaxis in the rat. Proc Natl Acad Sci USA 82, 1214-1218 (1985).
55. Scudamore, C. L., Thornton, E. M., McMillan, L., Newlands, G. F. & Miller, H. R Release of the mucosal mast cell granule chymase, rat mast cell protease-II, during anaphylaxis is associated with the rapid development of paracellular permeability to macromolecules in rat jejunum. J Exp Med 182, 1871-1881 (1995).
56. Yu, L. C. & Perdue, M. H. Role of mast cells in intestinal mucosal function: studies in models of hypersensitivity and stress. Immunol Rev 179, 61-73 (2001).
57. Bischoff, S. C. Mucosal allergy: role of mast cells and eosinophil granulocytes in the gut. Bail here's clinical gastroenterology 10, 443-459 (1996).
58. Gurish, M. F. & Austen, K. F. The diverse roles of mast cells. The Journal of experimental medicine 194. F1-5 (2001).
59. Galli, S. J., Maurer, M. & Lantz, C. S. Mast cells as sentinels of innate immunity. Curr.Opin. Immunol 11. 53-59 (1999).
60. Pejler, G., Abrink, M., Ringvall, M. & Wernersson, S. Mast cell proteases. Advances in immunology 95, 167-255, doi:S0065-2776(07)95006-3 [pii] 10.1016/S0065-2776(07)95006-3 [doi] (2007).
61. Bankova, L. G. et al. Mouse mast cell proteases 4 and 5 mediate epidermal injury through disruption of tight junctions. J Immunol 192, 2.812-2820, doi:10.4049/jimniunol.1301794 (2014).
62. Lawrence, C. E., Paterson, Y. Y., Wright, S. H., Knight, P. A. & Miller, H. R Mouse mast cell protease-1 is required for the enteropathy induced by gastrointestinal helminth infection in the mouse. Gastroenterology 127, 155-165, doi:S0016508504006092 [pii] (2004).
63. Groschwitz, K. R et al. Mast cells regulate homeostatic intestinal epithelial migration and barrier function by a chymase/Mcpt4-dependent mechanism. Proc Natl Acad Sci USA 106, 22381-22386, doi:0906372106 [pii] 10.1073/pnas.09063721.06 [doi] (2009).
64. Groschwitz, K. R.. Wu, D., Osterfeld, H., Ahrens, R. & Hogan, S. P. Chymase-mediated intestinal epithelial permeability is regulated by a protease-activating receptor/matrix metalloproteinase-2-dependent mechanism. Am J Physiol Gastrointest Liver Physiol 304, G479-489, doi:10.1152/ajpgi.00186.2012 (2013).
65. Jacob, C. et al. Mast cell tryptase controls paracellular permeability of the intestine. Role of protease-activated receptor 2 and beta-arrestins. J Biol Chem 280, 31936-31948 (2005).
66. Wilcz-Villega, E. M., McClean, S. & O'Sullivan, M. A. Mast cell tryptase reduces junctional adhesion molecule-A (JAM-A) expression in intestinal epithelial cells: implications for the mechanisms of barrier dysfunction in irritable bowel syndrome. The American journal of gastroenterology 108, 1140-1151, doi:10.1038/ajg.2013.92 (2013).
67. Scudamore, C. L. et al. Basal secretion and anaphylactic release of rat mast cell protease-H (RN/TP-11) from ex vivo perfused rat jejunum: translocation of RMCP-IT into the gut lumen and its relation to mucosal histology. Gut 37, 235-241 (1995)

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., such as) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law, Moreover, any combination of the above-described elements in all possible variations thereof is encompassed by the invention unless otherwise indicated herein or otherwise clearly contradicted by context.

1. A method comprising:
(a) obtaining a sample from a subject suspected of having a food allergy, and
(b) determining the presence of one or more fragments of the desmoglein-2 (DSG-2) protein using an immunoassay.

2. A method of treating a food allergy in a subject in need thereof, which method comprises:
(a) determining the presence of one or more fragments of the desmoglein-2 (DSG-2) protein in a sample obtained from the subject using an immunoassay; wherein the presence of one or more fragments of the DSG-2 protein indicates that the subject has a food allergy; and
(b) administering a therapeutic agent to the subject, whereby the food allergy is treated.

3. The method of claim 3, wherein the therapeutic agent is an antihistamine, epinephrine, oral immunotherapy, sublingual immunotherapy, or epicutaneous immunotherapy.

4. The method of any one of claims 1-3, further comprising determining the presence of full-length DSG-2 protein.

5. The method of any one of claims 1-4, which comprises determining the presence of a 22 kDa DSG-2 protein fragment, a 30 kDa DSG-2 protein fragment, and/or a 75 kDa DSG-2 protein fragment.

6. The method of any one of claims 1-5, wherein the immunoassay is selected from Western blot, radioimmunoassay, fluoroimmunoassay, enzyme-linked immunosorbent assay (ELISA), and chemiluminescent immunoassay.

7. The method of any one of claims 1-6, further comprising determining the presence of one or more fragments of an adherent junction (AJ) protein in the sample.

8. The method of any one of claims 1-7, further comprising determining the presence of one or more fragments of E-cadherin protein in the sample. The method of any one of claims 1-6, wherein the food allergy is selected from a peanut allergy, a tree nut allergy, a dairy allergy, a wheat allergy, a soy allergy, an egg allergy, a shellfish allergy, a meat allergy, and a corn allergy.

10. The method of any one of claims 1-9, wherein the sample is urine or blood.
